(19) United States
(12) Patent Application Publication
(10) Pub. No.: US 2014/0216994 A1
(43) Pub. Date: Aug. 7, 2014

(54) APPARATUS RELATING TO HEMODIALYSIS, HEMODIAFILTRATION, HEMOFILTRATION OR PERITONEAL DIALYSIS HAVING FUNCTION FOR RISE TEMPERATURE

(71) Applicant: Ho Youn Ki, Seoul (KR)

(72) Inventor: Ho Youn Ki, Seoul (KR)

(73) Assignee: WOODYOUNG MEDITECH CO., LTD., Seoul (KR)

(21) Appl. No.: 14/123,503

(22) PCT Filed: Jan. 3, 2012
(86) PCT No.: PCT/KR2012/000045

§ 371 Date: Apr. 9, 2014

(30) Foreign Application Priority Data
Jun. 2, 2011 (KR) 10-201100053149

(51) Int. Cl. A61M 1/36; A61M 1/28

(52) U.S. Cl. CPC A61M 1/369; A61M 1/28; A61M 2202/0021
USPC 210/97; 604/29

(57) ABSTRACT

Disclosed is an apparatus having a heating function for hemodialysis, hemodiafiltration, hemofiltration or peritoneal dialysis, wherein the apparatus includes at least one of pipe for transferring at least one fluid of blood and dialsate, and a heating unit for heating at least one fluid of blood and dialsate, wherein the fluids to be heated by the heating unit are substances to be injected into human body; and the heating unit is arranged for measuring flow rates of the fluids to be heated, and injecting temperatures related with the flow rate to heat the fluids to be heated. The heating unit comprises: flow passages through which the fluids to be heated are flowed; a heater formed as a part of the flow passages, for generating heat; and a cover means including a first connection portion through which the fluids enter the flow passages, and a second connection portion through which the fluids come out from the flow passages. Present invention provides one effect in that blood having the same or nearly same temperature as that of human body can be injected into human body to prevent side effects caused by the dialysis and another effect in that blood can be heated effectively because the shapes of flow passages around the heater is deformed in improved manner.

### TECHNICAL FIELD

The present invention relates to an apparatus having a heating function for hemodialysis, hemodiafiltration, hemofiltration or peritoneal dialysis, more specifically, to an apparatus for having a heating function for hemodialysis, hemodiafiltration, hemofiltration or peritoneal dialysis to provide a heating function by which temperature of blood or dialsate supplied to human body can be maintained to be similar to or the same as that of human body.

### BACKGROUND ART

When kidney in a human body has been functionally disordered in part or entire thereof, the human body loses functions for removing water and minerals, secreting noxious metabolite, maintaining acid-base equilibrium, and controlling concentrations of electrolyte and minerals within physiological ranges.

Such a disorder causes eventually blood of the human body to accumulate, in it, some bodily wastes including uremic waste metabolite such as urea, creatinine and uric acid, which, otherwise, would be all excreted through urin. Also, as a result, unbalance of electrolyte in the body may occur, which, in severe case, leads to death.

The hemodialysis and the peritoneal dialysis have been widely used as a substitute of the kidney function, in order to remove the above noxious bodily waste and excess water from the human body. In the hemodialysis and the peritoneal dialysis, the principles of diffusion and filtration are used to remove the noxious waste of blood from the human body and also to promote the balance of electrolyte.

The hemodialysis apparatus, typically, has a hemodialysis filter with dialysis membrane attached thereon to allow substances or materials to be moved and passed through the dialysis membrane between blood and dialsate, such that the bodily waste and toxins are dialyzed through semi-permeable dialysis membrane from the blood to dialsate, and excreted out of the body.

The dialysis membranes can be largely divided into flat membrane type and hollow fiber membrane type. However, nowadays, widely prevails the hemodials filter of hollow fiber membrane type in which a barrel-type container has bundles of hollow fiber membranes received therein and resin layers mounted on the both ends thereof.

In case of the peritoneal dialysis method, the hemodialysis filter is not required because the peritoneum of a patient itself is used as the dialysis membrane. More specifically, the peritoneal dialysis is a method in which a soft hose specially made is inserted into an abdominal region of the patient such that dialsate is injected and drained through the soft hose to remove bodily wastes, water and so forth from the human body.

FIG. 1 is a schematic view showing the structure of hemodialysis apparatus of general or typical type, according to prior art.

As shown in FIG. 1, the prior art hemodialysis apparatus includes a hemodialysis filter 100 for excreting the waste of blood with dialsate by allowing both the blood and the dialsate to pass through the hemodialysis filter 100, a pure dialsate tank 200 for supplying clean dialsate to the hemodialysis filter 100, a dialsate collection tank 300 for retaining dialsate previously passed through the hemodialysis filter 100, a balancer 400 for adjusting both feed and collection rates to be constant by comparing the amount of clean dialsate supplied to the hemodialysis filter 100 with the amount of dialsate collected from the hemodialysis filter 100, a blood pump 500 for supplying the blood of the patient to the hemodialysis filter 100, and a dialsate pump 600 for supplying the dialsate in the pure dialsate tank 200 to the hemodialysis filter 100.

The hemodialysis filter 100 includes a housing 110 having an interior space, and a dialysis membrane disposed the interior space of the housing 110. The housing 110 has a blood inlet 112 and a blood outlet 114 on upper and lower ends thereof, and a dials ate inlet 116 and a dials ate outlet 118 on lower and upper sides thereof, respectively. Accordingly, the blood enters into the blood inlet 112, goes through the midst of the dialysis membranes, and finally is excreted through the blood outlet 114, while the dials ate flows into the dials ate inlet 116, goes through a space between the dialysis membranes and the housing 110, and finally is excreted through the dialsate outlet 118.

In the blood dialysis filter 100, the blood and the dialsate flow forward in the opposite directions to each other. As the blood and the dialsate come closer to the blood outlet 114 and the dialsate outlet 118, respectively, the pressures of both the blood and the dialsate decrease gradually because the blood pump 500 and the dialsate pump 600 are disposed near the blood inlet 112 and the dialsate inlet 116, respectively,

More specifically, the pressure of the blood is higher than that of the dialsate in the upper portion of the housing 110, that is, an area on which the blood inlet 112 and the dials ate outlet 118 are formed, whereas the pressure of the dials ate is higher than that of the blood in the lower portion of the housing 110, that is, an area on which the blood outlet 114 and the dials ate inlet 116 are formed.

Accordingly, water, electrolyte and wastes are diffused into the dialsate, in the area where the pressure of the blood is higher than that of the dialsate, while the dialsate is transferred to the blood, in the area where the pressure of the dialsate is higher than that of the blood. As such a diffusion reaction continues, the wastes in the blood are gradually excreted out of the blood so that clean blood with the wastes removed can be supplied to the human body.

### CITATION LIST

Korean Patent Registration No. 10-1012535

### DISCLOSURE OF INVENTION

### Technical Problem

The above prior art hemodialysis apparatuses, however, have a disadvantage in that as the blood goes through the hemodialysis filter 100, the temperature of the blood goes continuously and gradually lower such that the temperature of blood has relatively much lower than that of human body, just at the time when the blood should be finally returned into the human body.

Generally, when the blood of lower temperature is injected into a human body, some energy produced by metabolism is required in order to increase the temperature of the injected blood to the same level as that of human body. As known well, the injected blood of lower temperature may decrease body heat and lead to heart shock and, in severe case, finally to dead.

Accordingly, it is required to heat the blood of lower temperature to the same or nearly same level as the temperature of the human body before the blood is injected to the human body.

The same applicant of present invention filed a Korean patent Application disclosing a medical heating apparatus in which fluids for therapy or blood is heated to the same or nearly same level as the human temperature previously before being injected into human body, and obtained the Letters Patent No. 10-1012535 thereof.

Present invention is intended to apply the medical heating apparatus to the apparatus for hemodialysis, hemodiafiltration, hemofiltration or peritoneal dialysis to prevent side effects caused by the hemodialysis.

Present invention is disclosed in order to solve the problems of prior arts based on the prior arts mentioned above. Specifically, the object of present invention is to provide an apparatus having a heating function for hemodialysis, hemodiafiltration, hemofiltration or peritoneal dialysis wherein means for heating blood or dials ate is added to general dialysis apparatuses of prior arts to prevent side effects caused by the dialysis.

Another object of present invention is to provide an apparatus having a heating function with a improved structure of flow passages, for hemodialysis, hemodiafiltration, hemofiltration or peritoneal dialysis wherein flow passages are formed diversely in shapes around a heater to allow blood and dialsate to be flowed smoothly and to heat blood and dialsate effectively.

However, all the objection of present invention is not limited to the above objects.

Other objects which are not described above can be apparently understood from the description mentioned below

### Solution to Problem

In order to attain the above object, one aspect according to the preferable embodiments of the present invention provides an apparatus having a heating function for hemodialysis, hemodiafiltration, hemofiltration or peritoneal dialysis, wherein the apparatus includes at least one of pipe for transferring at least one fluid of blood and dialsate, and a heating unit for heating at least one fluid of blood and dialsate, characterized in that the fluids to be heated by the heating unit are substances to be injected into human body; and the heating unit is arranged for measuring flow rates of the fluids to be heated, and injecting temperatures related with the flow rate to heat the fluids to be heated.

Preferably, the heating unit comprises: flow passages through which the fluids to be heated are flowed; a heater formed as a part of the flow passages, for generating heat; and a cover means including a first connection portion through which the fluids enter the flow passages, and a second connection portion through which the fluids come out from the flow passages.

More preferably, the cover means includes a first cover and a second cover, each of which has partition walls, respectively.

More preferably, the flow passages are defined by the heater disposed between the first cover and the second cover, and the first cover and the second combined with each other.

More preferably, the flow passages are defined by combining the partition walls formed on the front and rear surfaces of the heater, with the cover means.

More preferably, the flow passages are arranged to surround the heater so that the fluids can be moved, in spin, around the heater.

More preferably, the heater has resistance patterns formed on the front and rear surfaces thereof.

More preferably, the resistance patterns formed on the front and rear surfaces of the heater are electronically disconnected with each other.

More preferably, the heater is made of PCB(printed circuit board).

More preferably, the heater has a coating of parylene material applied on the front and rear surfaces thereof.

More preferably, a part of the heater is protruded out of the cover means, and a power applying electrodes are formed on the front and rear surfaces of the protruded part of the heater, respectively.

More preferably, temperature sensing electrodes connected with temperature sensors are formed on the front and rear surfaces thereof.

More preferably, the heater has through holes penetrating between the front and rear surfaces thereof, and the cover means are combined by the through holes.

More preferably, the apparatus further comprises a case comprised of an upper case and a lower case, in which the heating unit is received, and the case has power connectors and a electrode-inserting grooves into which a part of the heater is inserted.

Still more preferably, two power applying electrodes are formed on the front and rear surfaces of the heater, respectively, and the power of connectors are connected in parallel or serial through the power connectors.

Still further more preferably, the cover means are combined by one of UV bonding and ultrasonic bonding.

### Advantageous Effects of Invention

According to present invention, the apparatus having a heating function for hemodialysis, hemodiafiltration, hemofiltration or peritoneal dialysis provides an effect in that means for heating blood or dialsate is added to general dialysis apparatuses of prior arts so that blood or dialsate having the same or nearly same temperature as that of human body can be injected into human body to prevent side effects caused by the dialysis.

Furthermore, According to present invention, blood or dialsate can be heated effectively because the shapes of flow passages around the heater is deformed in improved manner.

### BRIEF DESCRIPTION OF DRAWINGS

The accompanying drawings, which are included to provide a further understanding of the invention and are incorporated in and constitute a part of this application, illustrate embodiment(s) of the invention and together with the description, and serve to explain the principle of the invention. In the drawings:

FIG. 1 is a schematic view illustrating schematically a structure of a typical hemodialysis apparatus, according to a prior art;

FIG. 2 is a schematic view illustrating schematically a structure of a hemodialysis apparatus having a heating function, according to one preferred embodiment of present invention;

FIG. 3 is an exploded perspective view illustrating a structure of a heating unit, according to one preferred embodiment of present invention;

FIG. 4 is an assembled perspective view illustrating a structure of a heating unit, according to one preferred embodiment of present invention;

FIG. 5 is a sectional view of line A-A of FIG. 4;

FIG. 6 is a perspective view illustrating a structure of a heating unit, according to another preferred embodiment of present invention;

FIG. 7 is a sectional view of line B-B of FIG. 6;

FIG. 8 is a sectional view illustrating a structure of a heating unit, according to another preferred embodiment of present invention;

FIG. 9 is an explanatory view illustrating connections between power applying electrodes and temperature sensing electrodes in the apparatus, according to one preferred embodiment of present invention;

FIGS. 10 and 11 are explanatory views illustrating relationships between electrical parallel and serial connections of power supply connectors in the heating unit of present invention;

FIGS. 12 is an explanatory view illustrating assembled status of the heating unit of present invention; and

FIGS. 13 is a flow chart illustrating operating of present invention.

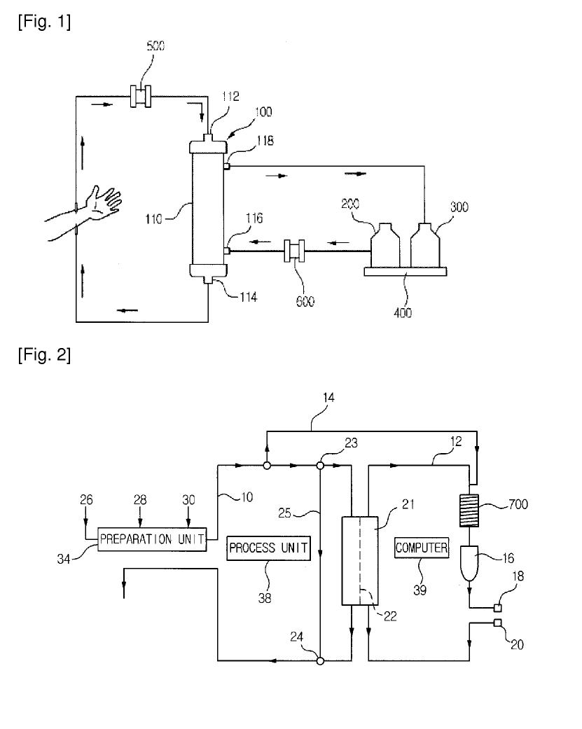

### REFERENCE SIGNS LIST

10: first fluid circuit

12: second fluid circuit,

14: pipe,

16: drip chamber,

18 and 20: connecting portions,

21: blood filter,

22: semi-permeable dialysis membrane,

23 and 24: valves,

25: bypass pipe

26, 28 and 30: inlets,

34: preparing unit,

38: process unit,

39: computer,

700: heating unit,

710: case,

711: lower case, 712: upper case

714a and 714b: power connectors,

720: first cover,

721: first connection portion,

727: first partition walls,

740: second cover,

747: second partition walls,

750: heater,

752: resistance patterns,

753: power applying electrodes,

754: temperature sensing electrodes,

756: through holes

757: temperature sensors,

760: partition wall member,

### BEST MODE FOR CARRYING OUT THE INVENTION

Hereinafter, the present invention according to embodiments will be described in more detail with reference to the accompanying drawings.

The embodiments described below are mainly based on the hemodialysis apparatus. The gist or technical ideal, however, of present invention can be also applied to all the hemodiafiltration apparatus, hemofiltration apparatus or peritoneal dialysis apparatus.

The embodiments described below with reference to the accompanying drawings, are described only for better understanding of characteristics of present invention, and are not intended to limit the scope of present.

Further, in case that description of well-known functions or structures related to present invention may interfere to understand the gist of present invention, the description will be omitted for simplicity.

Furthermore, for efficient description of technically structural elements, the descriptions about the already existing elements or the typically well-known existing elements will be omitted as possible. In other words, the descriptions of the embodiments described hereinafter are based mainly on newly added elements for present invention.

FIG. 2 is a schematic view illustrating schematically a structure of a hemodialysis apparatus having a heating function according to one preferred embodiment of present invention;

According to one preferred embodiment of present invention, a hemodialysis apparatus having a heating function includes a first fluid circuit 10 for dialsate, a second fluid circuit 12 for blood, and also a pipe 14 for directly injecting the dialsate into human body, as shown in FIG. 2.

The second fluid circuit 12 includes a drip chamber 16 which is connected to the pipe 14. Connecting portions 18 and 20 is connected with a patient. A blood filter 21 is connected between the first fluid circuit 10 and the second fluid circuit 12. The blood filter 21 has a semi-permeable dialysis membrane 22 therein.

On the contrary, according to another embodiment of present invention, the apparatus for peritoneal dialysis does not have dialysis device or the blood filter 21 because the peritoneum of a patient itself functions as a dialysis membrane.

A bypass pipe 25 is connected between valves 23 and 24. Accordingly, dialsate can be passed through the bypass pipe 25 instead of the blood filter 21 by properly setting the valves 23 and 24.

According to the embodiment of present invention, a plurality of inlets 26, 28 and 30 for injecting distilled water or dialsate are formed. The numbers of inlets may vary optionally and necessarily.

An exact composition of dialsate can be prepared in a preparing unit 34.

Referential numerals 38 and 39 unspecified up to now indicate process unit and a computer controlling general operation of the apparatus, respectively. An exact composition of dialsate can be prepared in a preparing unit 34.

In the dialysis apparatus described above, the description of well-known elements and their functions is omitted for simplicity.

The structures of the apparatus described above up to now are well-known by an ordinary person in the art.

A referential numeral 700 indicates a heating unit for heating at least one fluid of blood and dialsate, which is one of characteristics of present invention. The heating unit 700 heats fluids such as blood and dialsate which are heated preferably just before being injected into human body. In the heating unit 700, the energy used for heating depends on a flow rate of fluid to be heated, and an injection temperature for dialysis. Accordingly, the heating unit 700 is arranged to effectively heat the fluids to be heated based on the flow rates and the injection temperatures which may vary.

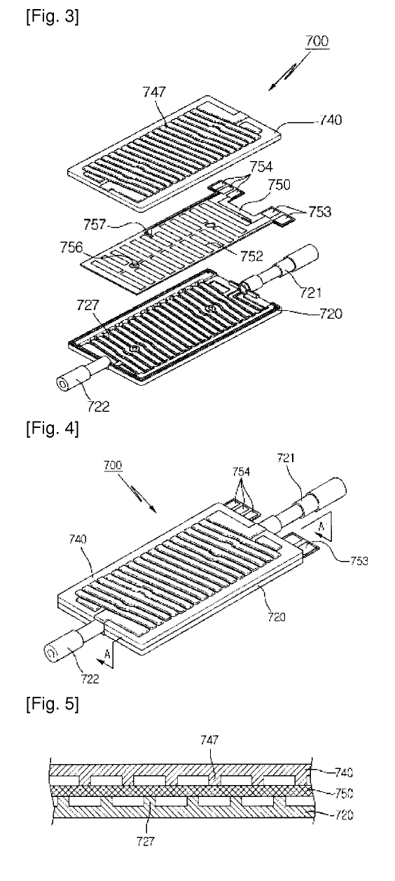

As shown in FIGS. 3 and 5, the heating unit 700 according to present invention includes a first cover 720, a heater 750 and a second cover 740. The heater 750 is disposed between the first cover 720 and the second cover 740 which are combined with each other by one of UV bonding and ultrasonic bonding.

A plurality of first partition walls 727 are formed on a surface of the first cover 720 opposite to the heater 750, while a plurality of second partition walls 747 are formed on a surface of the second cover 740 opposite to the heater 750. The first cover 720 and the first partition walls 727 are integrally formed by projection extrusion, also, the second cover 740 and the second partition walls 747 are integrally formed by injection molding.

The first partition walls 727 and the second partition walls 747 are all inclined such that the walls 727 and 747 together with the heater 750 form continuous flow passages.

Substantial U shapes are formed between partition walls in the first cover 720 and the second cover 740, respectively. Therefore openings of the U shapes are closely contacted with the heater 750 to form “□” shapes of flow passages in sectional view. In this case, the flow passages of “□” shapes are formed on both front and rear surfaces of the heater 750 such that the directions of the flow passages run in the width direction of the heater 750 covered by the passages, as shown in FIG. 3. However, the flow passages may run in the length direction of the heater, for better efficient heating.

The flow passages are formed like shape of a thread screw surrounding the heater 750, by one combination of the front surface of the heater 750 and the first cover 720 including the first partition walls 727, and another combination of the rear surface of the heater 750 and the second cover 740 including the second partition walls 747 such that the flow passages are extended along with the front and the rear surfaces of the heater 750 to form a tube shape of passages.

The heater 750 forms surfaces of the flow passages such that heat generated from the heater 750 not only can be efficiently transferred to the fluids flowing through the flow passages, but also can allow the blood injected at higher feed rate to flow smoothly, and therefore prevent red blood cells and etc., in blood from being damaged.

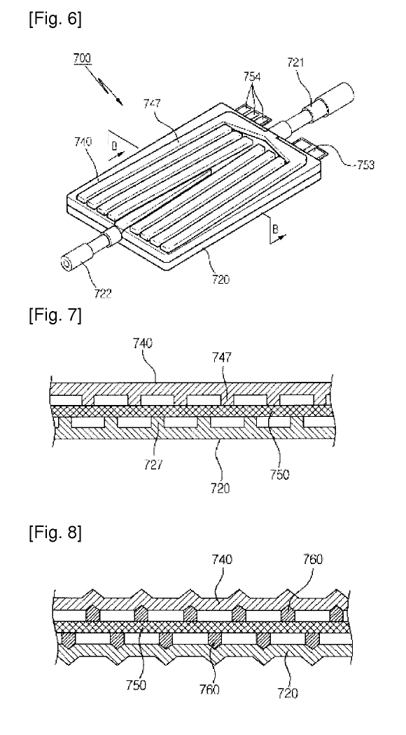

Furthermore, according to another embodiment of present invention, the heater 750 may have partition wall members 760 combined with the first cover 720 and the second cover 740 such that flow passages having the thread screw shape are extended in the directions of both width and length of the heater, as shown in FIG. 8. In other words, in this embodiment, the partition wall members 760 are not formed on the first cover 720 and the second cover 740, but formed on the heater 750.

Also, the first cover 720 includes a first connection portion 721 into which the fluids enter, and a second connection portion 722 through which the fluids go out. Therefore, the fluids, firstly, enter through the first connection portion 721, pass through the flow passages and finally go out through the second connection portion 722, based on the combination of the first cover 720 including the first partition walls 727, and the second cover 740 including the second partition walls 747.

The heater 750 has resistance patterns 752 on the both the front and the rear surfaces thereof. The resistance patterns 752 are connected to power applying electrodes also disposed on the both the front and the rear surfaces of the heater 750, so that the resistance patterns 752 can produce heat with power energy applied by the power applying electrodes 753.

The heater 750 may be made in the various types of plates. However, it is more preferable that the heater 750 is made of PCB(Printed Circuit Board) through which precision resistance values of the resistance patterns and mass production of the heater with low cost can be attained.

In case that the resistance patterns 752 formed on the front and rear surfaces of the heater 750 are connected with each other in serial or parallel, via holes should be used to connect the resistance patterns 752. The via holes, however, tend to cause the resistance values to be, in adverse effect, changed during via hole forming process. In order to prevent such a side effect caused by via holes, it is preferable that the resistance patterns 752 are not electrically connected with each other, of course, without using the via holes.

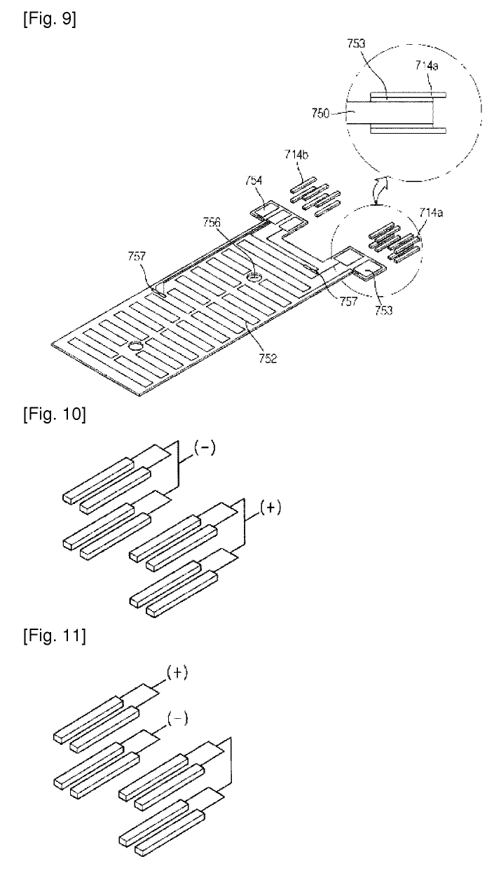

In order to obtain desired heat amount from the heater 750, it is required to supply proper amount of power to the heater 750 and sense the temperature of the heater through temperature sensors. More specifically, it is required to connect the resistance patterns 752 with the temperature sensors 757 formed on the front and rear surfaces of the heater, in serial or parallel without via holes, for the reason mentioned above. For this purpose, as shown in FIGS. 9 and 11, power connectors 714a and 714b are used instead of via holes.

On the front and rear surfaces of the heater 750, there are the power applying electrodes 753, temperature sensing electrodes 754 and the temperature sensor 757 which are all connected in parallel (FIG. 10) and in serial (FIG. 11) through the power connectors 714a and 714b.

Furthermore, in order to improve dielectric strength, waterproof and surface lubricity, and prevent that harmful substances which may be produced on the surfaces of the heater 750 contacting with blood or dialsate are injected into human body, it is preferably that a coating of Poly-Para-Xylylene, Parylene material is applied on the front and rear surfaces of the heater 750.

Still furthermore, as shown in FIG. 4, a part of the heater 750 is protruded out of the first cover 720 and the second cover 740. The power applying electrodes 753 and the temperature sensing electrodes 754 are formed on the front and rear surfaces of the protruded part of the heater 750.

Generally, the fluids tend to have more air in the higher temperature thereof than lower temperature. For this, the first cover 720 and the second cover 740 may further include an air filter portion (not shown) on the flow passages for removing air. For example, the air filter portion may have a membrane having fine gaps, of which material is hydrophobic or not-fluids-affinity.

Further, the heater 750 may have the temperature sensor 757 on the front and rear surfaces thereof for sensing temperature of the fluids to send the sensed signal to the outside via the temperature sensing electrodes 754.

Furthermore, the heater 50 has through holes 756 penetrating between the front and rear surfaces thereof, for tightly and closely fastening the first partition walls 727 of the first cover 720 and the second partition walls 747 of the second cover 740 to the surfaces of the heater 750 to force the powerful physical contacts and assembly between the walls 727 and 747 and the surfaces of the heater 750. Accordingly, the heating unit of present invention can endure higher interior pressures which may occur upon injecting of the fluids at high feed rate. Therefor, the through holes provide an assistant effect for the UV bonding or the ultrasonic bonding.

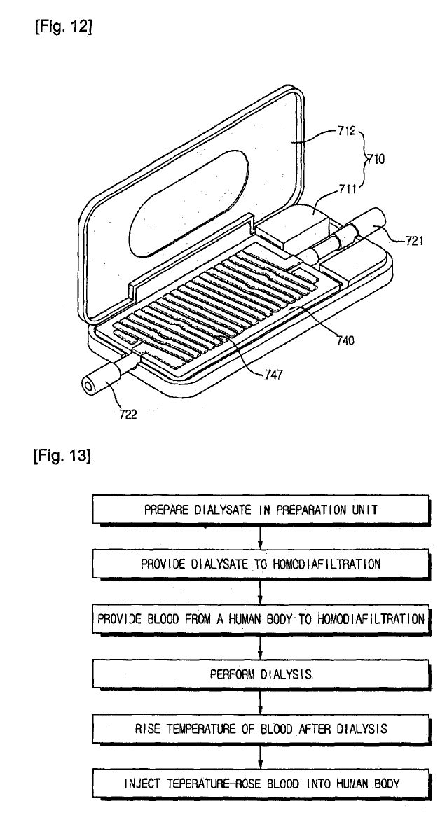

As shown in FIG. 12, the apparatus of present invention further includes a case 710 having electrode-inserting grooves for receiving the electrodes which are part of the heater 750. Power connectors for connecting the power applying electrodes 753 and the temperature sensing electrodes 754 formed in the front and rear surfaces of the heater 750 in parallel or serial are formed on the upper and lower sides of the electrode-inserting grooves.

The case includes a lower case 711 and a upper case 712 which are combined with each other. The heating unit 700 is disposed between the cases 711 and 712.

Hereinafter, the operation of the hemodialysis apparatus having the structure mentioned above will be described.

As shown in FIG. 13, general operations of the apparatus of present invention are controlled by the process unit 38 or the computer 39.

For hemodialysis, above all, the dials ate is supplied to the blood filter 21 through preparing unit 34, and via the first fluid circuit 10, while the blood is also supplied to the blood filter 21 through the connecting portion 20 connected with a human body of a patient, and via the second fluid circuit 12.

In the blood filter 21, impurities in the blood are excreted to the dialsate by a pressure difference between the blood and the dialsate. The clean blood in which the impurities have been already removed is supplied to the human body through the second fluid circuit 12, the heating unit 700 and the drip chamber 16, while the supplied blood have the same or nearly same temperature as that of human body by heating of the blood.

It will be apparent to those of ordinary skill in the art that various modifications can be made to the exemplary embodiments of the invention described above. However, as long as modifications fall within the scope of the appended claims and their equivalents, they should not be misconstrued as a departure from the scope of the invention itself.

### INDUSTRIAL APPLICABILITY

As mentioned above, according to present invention, the apparatus having a heating function for hemodialysis, hemodiafiltration, hemofiltration or peritoneal dialysis provides an effect in that means for heating blood or dialsate is added to general dialysis apparatuses of prior arts so that blood or dialsate having the same or nearly same temperature as that of human body can be injected into human body to prevent side effects caused by the dialysis.

1. An apparatus having a heating function for hemodialysis, hemodiafiltration, hemofiltration or peritoneal dialysis, wherein the apparatus includes at least one of pipe for transferring at least one fluid of blood and dialsate, and a heating unit for heating at least one fluid of blood and dialsate, characterized in that
the fluids to be heated by the heating unit are substances to be injected into human body; and
the heating unit is arranged for measuring flow rates of the fluids to be heated, and injecting temperatures related with the flow rate to heat the fluids to be heated.

2. The apparatus having a heating function for hemodialysis, hemodiafiltration, hemofiltration or peritoneal dialysis, according to claim 1, wherein the heating unit comprises:
flow passages through which the fluids to be heated are flowed;
a heater formed as a part of the flow passages, for generating heat; and
a cover means including a first connection portion through which the fluids enter the flow passages, and a second connection portion through which the fluids come out from the flow passages.

3. The apparatus having a heating function for hemodialysis, hemodiafiltration, hemofiltration or peritoneal dialysis, according to claim 2, wherein the cover means includes a first cover and a second cover, each of which has partition walls, respectively.

4. The apparatus having a heating function for hemodialysis, hemodiafiltration, hemofiltration or peritoneal dialysis, according to claim 2 or 3, wherein the flow passages are defined by the heater disposed between the first cover and the second cover, and the first cover and the second combined with each other.

5. The apparatus having a heating function for hemodialysis, hemodiafiltration, hemofiltration or peritoneal dialysis, according to claim 2, wherein the flow passages are defined by combining the partition walls formed on the front and rear surfaces of the heater, with the cover means.

6. The apparatus having a heating function for hemodialysis, hemodiafiltration, hemofiltration or peritoneal dialysis, according to claim 2, wherein the flow passages are arranged to surround the heater so that the fluids can be moved, in spin, around the heater.

7. The apparatus having a heating function for hemodialysis, hemodiafiltration, hemofiltration or peritoneal dialysis, according to claim 2, wherein the heater has resistance patterns formed on the front and rear surfaces thereof.

8. The apparatus having a heating function for hemodialysis, hemodiafiltration, hemofiltration or peritoneal dialysis, according to claim 2 or 7, wherein the resistance patterns formed on the front and rear surfaces of the heater are electronically disconnected with each other.

9. The apparatus having a heating function for hemodialysis, hemodiafiltration, hemofiltration or peritoneal dialysis, according to claim 2, wherein the heater is made of PCB (printed circuit board).

10. The apparatus having a heating function for hemodialysis, hemodiafiltration, hemofiltration or peritoneal dialysis, according to claim 2, wherein the heater has a coating of parylene material applied on the front and rear surfaces thereof.

11. The apparatus having a heating function for hemodialysis, hemodiafiltration, hemofiltration or peritoneal dialysis, according to claim 2, wherein a part of the heater is protruded out of the cover means, and a power applying electrodes are formed on the front and rear surfaces of the protruded part of the heater, respectively.

12. The apparatus having a heating function for hemodialysis, hemodiafiltration, hemofiltration or peritoneal dialysis, according to claim 2, wherein temperature sensing electrodes connected with temperature sensors are formed on the front and rear surfaces thereof.

13. The apparatus having a heating function for hemodialysis, hemodiafiltration, hemofiltration or peritoneal dialysis, according to claim 2, wherein the heater has through holes penetrating between the front and rear surfaces thereof, and the cover means are combined by the through holes.

14. The apparatus having a heating function for hemodialysis, hemodiafiltration, hemofiltration or peritoneal dialysis, according to claim 2, wherein the apparatus further comprises a case comprised of an upper case and a lower case, in which the heating unit is received, and the case has power connectors and a electrode-inserting grooves into which a part of the heater is inserted.

15. The apparatus having a heating function for hemodialysis, hemodiafiltration, hemofiltration or peritoneal dialysis, according to claim 11, wherein two power applying electrodes are formed on the front and rear surfaces of the heater, respectively, and the power of connectors are connected in parallel or serial through the power connectors.

16. The apparatus having a heating function for hemodialysis, hemodiafiltration, hemofiltration or peritoneal dialysis, according to claim 2, wherein the cover means are combined by one of UV bonding and ultrasonic bonding.
